(19) United States
(12) Patent Application Publication
(10) Pub. No.: US 2025/0288254 A1
(43) Pub. Date: Sep. 18, 2025

(54) Imaging Modality with Patient Positioning Specification

(71) Applicant: Siemens Healthineers AG, Forchheim (DE)

(72) Inventors: Johann Sukkau, Herzogenaurach (DE); Julian Wohlers, Erlangen (DE)

(73) Assignee: Siemens Healthineers AG, Forchheim (DE)

(21) Appl. No.: 19/081,138

(22) Filed: Mar. 17, 2025

(30) Foreign Application Priority Data
Mar. 18, 2024 (DE) 10 2024 202 519.4

(51) Int. Cl. A61B 5/00; A61B 5/055; A61B 6/00; A61B 6/04

(52) U.S. Cl. CPC A61B 5/704; A61B 5/055; A61B 6/0407; A61B 6/54

(57) ABSTRACT

A medical imaging system includes a longitudinally movable patient couch that positions different patient regions into its isocenter. A control facility receives patient data—including arrangement on the couch—and an operator's selection of the region to be examined. Based on these inputs, it provisionally identifies the couch section where the selected region is located and projects a marking there. The operator can issue correction commands to shift the marking until giving a close command. At that point, the system accepts the current location of the marking as final and moves the couch so that this section is in the isocenter.

### BACKGROUND

Independent of the grammatical term usage, individuals with male, female, or other gender identities are included within the term.

The present disclosure is based on an operating method for a medical imaging system, wherein the system has a patient couch which can be moved in a longitudinal direction, so that by moving the patient couch in the longitudinal direction different regions of a patient lying on the patient couch can be moved in the longitudinal direction into an isocenter of the medical imaging system,
wherein a control facility of the system receives from an operator data which characterizes properties of the patient him/herself and the approximate arrangement of the patient on the patient couch,
wherein the control facility receives a selection command from the operator for a selection of a region of the patient which is to be examined by means of the medical imaging system,
wherein the control facility utilizes the patient's data to determine a section of the patient couch, viewed in the longitudinal direction of the patient couch,
wherein the control facility moves the patient couch such that the section is moved in the longitudinal direction into the isocenter of the medical imaging system.

The present disclosure is further based on a control program for a software-programmable control facility for a medical imaging system, wherein the control program comprises machine code which can be processed directly by the control facility, wherein the processing of the machine code by the control facility causes the control facility to execute such an operating method.

The present disclosure is further based on a control facility for a medical imaging system, wherein the control facility is programmed with such a control program so that the control facility executes such an operating method.

The present disclosure is further based on a medical imaging system,
wherein the system has a patient couch that can be moved in a longitudinal direction so that by moving the patient couch in the longitudinal direction, different regions of a patient lying on the patient couch can be moved in the longitudinal direction into an isocenter of the medical imaging system,
wherein the system has an interface to an operator,
wherein the system has a control facility with a data connection to the patient couch and the interface,
wherein the control facility is designed as such a control facility which controls the medical imaging system in accordance with such an operating method.

The above-mentioned subject matters are generally known. In particular, the subject matters mentioned are employed in connection with what is known as the APO method.

In the APO method, the operator of the control facility specifies relevant data about the patient, for example, the age, height, sex, and weight of the patient as well as the approximate arrangement of the patient on the patient couch. The data for the characterization of the approximate arrangement of the patient on the patient couch can include information as to whether the patient is lying feet or head first on the patient couch. Furthermore, the data for the characterization of the approximate arrangement of the patient on the patient couch can include whether the patient is lying on his/her back, stomach, or left or right side. Other information is also possible (legs extended or bent, Superman position, etc.). The operator then selects an organ from a list, for example, the heart, liver, or lungs. Based on a modeling of the patient, which is parameterized using the relevant data about the patient, the control facility then determines the section of the patient couch which is to be moved into the isocenter and moves the patient couch accordingly.

A fundamental problem of the APO method is that the modeling of the patient deviates from the real patient, nor is the location of the patient on the patient couch specified exactly. For example, the patient may lie a little higher or a little lower on the patient couch, even if he/she is, for example, lying feet first on the patient couch according to the information. This results in a deviation of the movement of the patient couch from the actually desired movement of the patient couch, which can be in the region of several centimeters. Deviations of 2 cm to 15 cm are typical. The deviation of the movement of the patient couch from the actually desired movement is not identified until after a number of images of the region of the patient have been captured by means of the medical imaging system and output to the operator. Only then is the control facility given positioning commands by the operator, on the basis of which it moves the patient couch in the longitudinal direction and thus corrects the positioning in the longitudinal direction.

In an earlier patent application by the applicant which was not published in advance—this application does not represent any generally known system—it has already been proposed to assign the medical imaging system a display facility actuated by the control facility, by means of which a marking is projected onto a section of the patient couch. The location onto which the marking is projected can then be relocated by the operator by specifying correction commands. If the marking is relocated by the operator into the section desired by said operator, the latter specifies a close command. Due to the close command, the control facility moves the patient couch such that the most recently marked section is moved in the longitudinal direction into the isocenter of the medical imaging system.

### SUMMARY

An object of the present disclosure is to create opportunities by means of which the known APO method can be improved.

In accordance with the disclosure, an operating method of the type mentioned in the introduction is configured, in that
the section determined by the control facility utilizing the data about the patient is only provisional, i.e., at this section the selected region of the patient is only provisionally assumed,
the control facility actuates a display facility so that a marking is projected onto the provisionally determined section of the patient couch,
on the basis of correction commands that it receives from the operator, the control facility relocates the marking in the longitudinal direction until it receives a close command from the operator,
On the basis of the specification of the close command, the control facility accepts as final the section onto which the marking is projected during specification of the close command, and the section that is moved in the longitudinal direction into the isocenter of the medical imaging system is the final section.

Thus, with the procedures described in the above-mentioned earlier patent application of the applicant, which was not published in advance, there is an improvement in the APO process.

Just as in the known system, the control facility can be provided by the operator with properties of the patient, for example, the age, height, sex, and weight of the patient. The approximate arrangement of the patient can consist of specifying whether the patient is lying feet or head first on the patient couch. Furthermore, it can, for example, also be specified whether the patient is lying on his/her back, stomach, or left or right side. Other specifications are also possible (legs extended or bent, Superman position, etc.).

The correction commands can, if required, be absolute in nature (“the marking should be positioned here”) or relative in nature (“the marking should be relocated in this direction”).

In the simplest case, the marking (merely) comprises a line running transversely to the longitudinal direction of the patient couch. This procedure is simple and reliable. To achieve a line running transversely, it is possible for the display facility to be moved to the corresponding position in the longitudinal direction of the patient couch, following which the line is projected, for example, by means of a laser and a pivotable deflection mirror. However, a procedure has already also been explained in another of the applicant's patent applications, likewise not published in advance—this application too does not represent any generally known system—in which no movement of the display facility is necessary, but the relocations and displacements of the line which are caused by the contour of the patient are compensated for by corresponding beam guidance.

Alternatively or additionally, to project the line, it is possible for the marking to comprise an image of the selected region of the patient. It is possible for just one generic image (for example, the typical contour of a liver or a lung) to be projected. However, it is particularly preferred if the control facility ascertains the size of the image (and possibly also the location of the image) as a function of the data characterizing the properties of the patient him/herself.

The specification of the correction commands can take place as required. For example, the control facility can receive the correction commands as voice commands, as gestures, or as manual inputs. The specification can be configured highly intuitively, for example, in the case of voice commands by the voice commands “forward”, “back,” and “stop”, where appropriate supplemented by the voice commands “slower” and “faster”. In the case of gestures, it is possible to point with the finger or the whole arm in the desired direction (relative relocation) or at the desired position (absolute positioning). Corresponding identifications using one or more cameras are known as such and, as such, are not the subject of the present disclosure. A manual specification, for example, via arrow keys of a keyboard or corresponding marked regions of a touch screen or via a wheel that is rotated forward or backward as required, is likewise possible.

The specification of the close command can be explicit in nature, for example, in the case of voice commands, the word “ready”. In the case of gesture control, the arms can be crossed, for example. In the case of an input via an interface, there are many readily known possibilities. However, it is also possible for the specification of the close command to be implicit in nature. In particular, a lapse of time (“no change during the last x seconds”) can, where appropriate, be interpreted as a close command. However, regardless of the way in which the close command is specified, it is preferably provided that due to the specification of the close command, the control facility terminates the actuation of the display facility.

In connection with the provisional determination of the section of the patient couch, the control facility normally utilizes not only the data about the patient but also a model of the patient, which is parameterized with parameters. The parameters can, for example, be derived from a database in which a plurality of datasets are stored. The datasets can, for example, contain anonymized data for a patient in each case, the properties of the respective patient, and the location of the selectable regions of this patient. The control facility preferably tracks at least one of the parameters of the model utilizing the location of the final section of the patient couch or the difference between the provisionally determined section of the patient couch and the final section of the patient couch. For example, an additional dataset of the now concrete patient can be stored in the database, wherein this dataset for the examined region is assigned the concrete value at which the patient couch is finally positioned.

Normally, after the patient couch is moved such that the (final) section is moved in the longitudinal direction into the isocenter of the medical imaging system, the control facility captures a number of images of the region of the patient by means of the medical imaging system and outputs the images to the operator. The control facility can then receive positioning commands from the operator, on the basis of which it moves the patient couch in the longitudinal direction. This procedure is known as the capture of localizer images together with positioning tracking.

Here, too, it is possible for the control facility to track at least one of the parameters of the model, utilizing the extent to which it moves the patient couch in the longitudinal direction on the basis of the positioning commands or the point to which it moves the patient couch in the longitudinal direction. The procedure is, in essence, analogous to tracking the model on the basis of the location of the final section of the patient couch or the difference between the provisionally determined section of the patient couch and the final section of the patient couch.

In accordance with the disclosure, a medical imaging system of the type mentioned in the introduction is configured, such that
the system is assigned a display facility, by which a marking can be projected onto a section of the patient couch,
the control facility also has a data connection to the display facility and
the control facility is designed as an inventive control facility which controls the medical imaging system in accordance with an inventive operating method.

### DESCRIPTION OF THE DRAWINGS

The above-described properties, features, and advantages of this disclosure and the manner in which they are achieved will become clearer and more readily understandable in connection with the following description of the exemplary aspects, which are explained in greater detail in connection with the drawings. These are shown schematically:

FIG. 1 illustrates a medical imaging system from the side,

FIG. 2 illustrates the medical imaging system from FIG. 1 from above,

FIG. 3 illustrates a flow diagram,

FIG. 4 illustrates a patient couch and a marking from above,

FIG. 5 illustrates a partial view of the medical imaging system from FIGS. 1 and 2 from the side,

FIG. 6 illustrates configurations of an interface, and

FIG. 7 illustrates a flow diagram.

### DETAILED DESCRIPTION

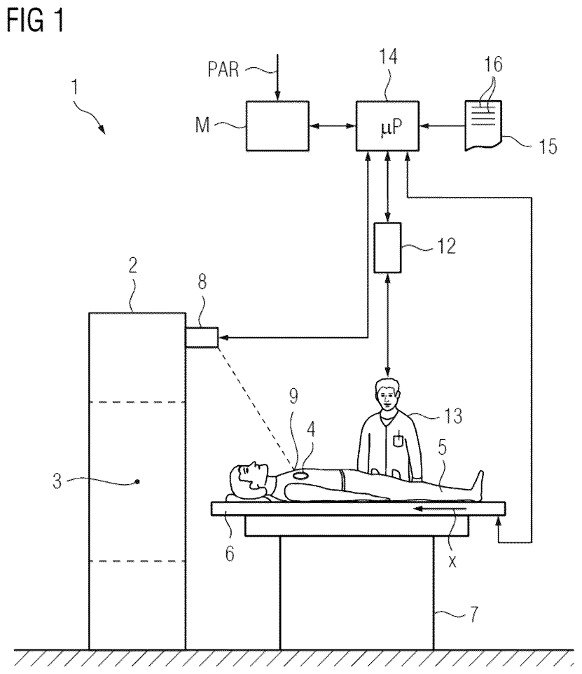

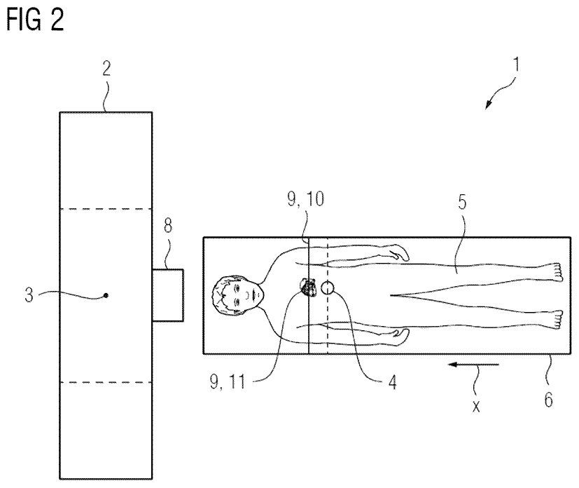

In accordance with FIGS. 1 and 2, a medical imaging system 1—referred to below only for short as a modality 1—has a base body 2. The base body 2 defines an isocenter 3, i.e., the region of the modality 1 in which a region 4 of an object under examination 5 is arranged if the corresponding region 4 is to be examined by means of the modality 1.

The modality 1 can, for example, be a magnetic resonance system. Modality 1 can, however, also be configured differently, for example, as a computed tomography system or as a C-arm X-ray system. Other configurations, for example, as MR-PET, PET-CT, and PET, are possible. The object under examination 5 is generally a person, also referred to below as a patient. Furthermore, in FIGS. 1 and 2, it is assumed purely by way of example that the heart of a patient 5 is the region 4, which is to be examined.

To be able to position the patient 5 properly, the modality 1 has a patient couch 6. The patient couch 6 is generally arranged on a substructure 7. The patient couch 6 can be moved in a longitudinal direction x by means of a corresponding drive. By moving the patient couch 6 in the longitudinal direction x different regions 4 of the patient 5 can be moved in the longitudinal direction x into the isocenter 3. For this, patient 5 must, of course, be lying on the patient couch 6.

The modality 1 is further assigned a display facility 8. The display facility 8 can theoretically be arranged on the base body 2. The display facility 8 is often arranged on the ceiling of a room in which the modality 1 is arranged. By means of the display facility 8, it is possible to project a marking 9 onto a section of the patient couch 6. The marking 9 can, for example, comprise a line 10 running transversely to the longitudinal direction x. Alternatively or additionally, the marking 9 can, as indicated highly symbolically in FIG. 2, comprise an image 11 of the region 4.

The display facility 8 can, if required, be stationary or can be moved in the longitudinal direction x and arranged on the base body 2 or the ceiling. The display facility 8 can, for example, be designed as a normal projector or as a laser.

The modality 1 further has an interface 12 to an operator 13. The interface 12 has a data connection to a control facility 14 of the modality 1. The operator 13 can communicate with the control facility 14 via the interface 12. The control facility 14 controls (inter alia) the movement of the patient couch 6 and the display facility 8. The control facility 14 hence also has a data connection to the patient couch 6 and the display facility 8.

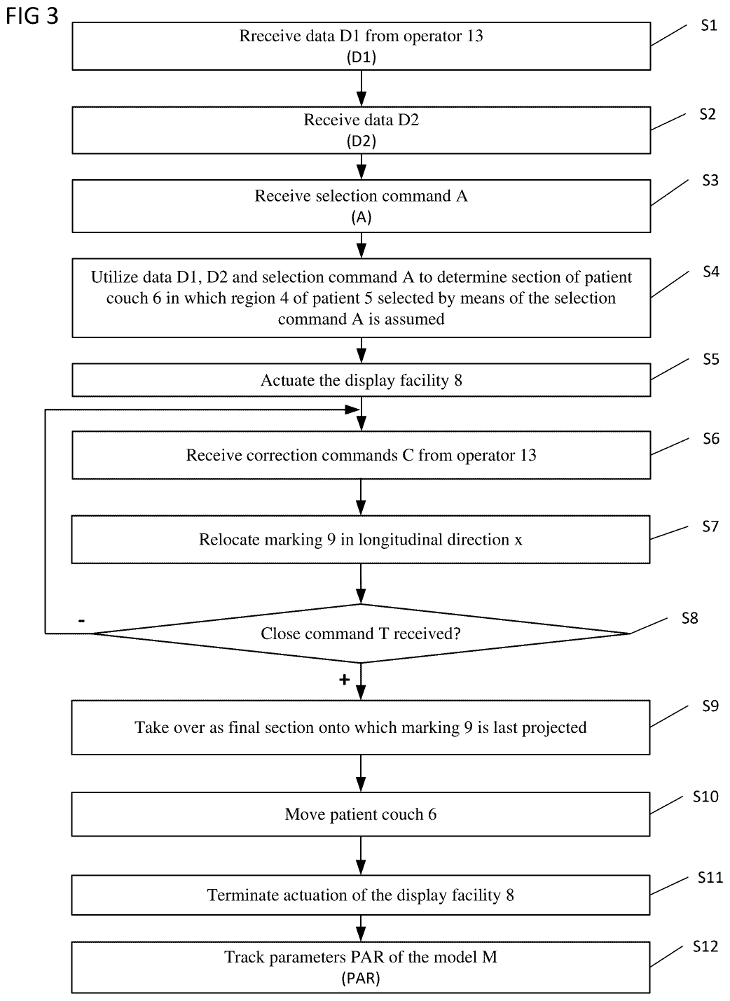

The control facility 14 is microprocessor-controlled, as indicated in FIG. 1 by “μP”, and is thus designed as a software-programmable facility. The operating principle of the control facility 14 is thus fixed by a control program 15, with which the control facility 14 is programmed. The control program 15 comprises machine code 16, which can be processed directly by the control facility 14. The processing of the machine code 16 by the control facility 14 (in other words, the programming of the control facility 14 with the control program 15) causes the control facility 14 to execute an operating method which is explained in greater detail below in connection with FIG. 3. By executing this operating method the modality 1 is controlled accordingly.

In accordance with FIG. 3 the control facility 14 receives data D1 from the operator 13 in a step S1. The data D1 characterizes the patient 5 him/herself. The data D1 can, for example, comprise the height, weight, age, and sex of the patient 5. Furthermore, in a step S2, the control facility 14 receives data D2. The data D2 characterizes the approximate arrangement of the patient 5 on the patient couch 6, for example, whether the patient 5 is lying head or feet first on the patient couch 6. Furthermore, in a step S3, the control facility 14 receives a selection command A. By means of the selection command A, a selection is made as to which region 4 of the patient 5 is to be examined by means of the modality 1. The specifications of steps S1 to S3 take place via the interface 12.

In a step S4, the control facility 14 utilizes the data D1, D2, and the selection command A to determine the section of the patient couch 6 in which, viewed in the longitudinal direction x, the region 4 of the patient 5 selected by means of the selection command A is assumed. Normally, the control facility 14 utilizes a model M of the patient 5 in addition to the data D1, D2 in connection with step S4. The model M can be parameterized in accordance with FIG. 1, in particular with parameters PAR, wherein the parameters PAR can be determined by utilizing the data D1, D2. In a step S5, the control facility 14 actuates the display facility 8. The actuation takes place such that the marking 9 is projected onto the section of the patient couch 6 determined in step S4. FIG. 2 shows purely by way of example the marking 9 in solid lines.

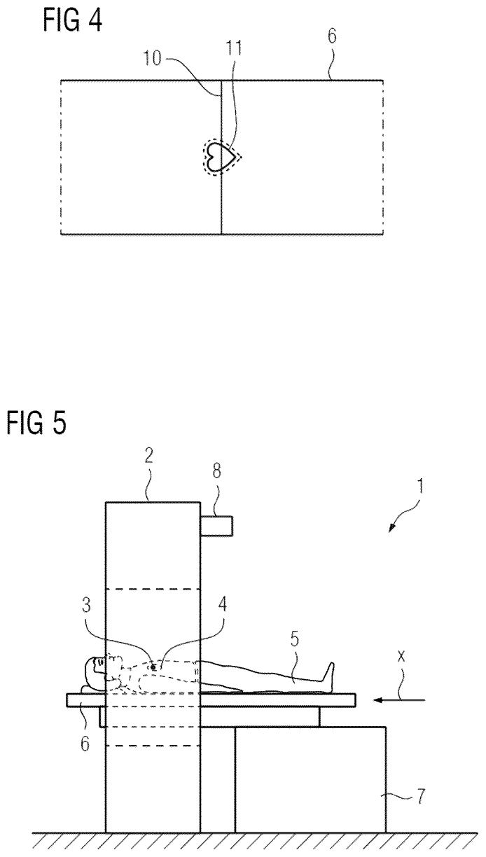

If the marking 9 comprises an image 11 of the region 4, it is possible—see the representation in solid lines in FIG. 4—that the image 11 of the region 4 is always one and the same generic image. Alternatively, it is possible that the control facility 14—see the representation in broken lines in FIG. 4—ascertains the size of the image 11 as a function of the data D1. For reasons of clarity, the patient 5 is not represented in FIG. 4.

In a step S6, the control facility 14 receives correction commands C from the operator 13. On the basis of the correction commands C, the control facility 14 relocates the marking 9 in the longitudinal direction x in a step S7. For example, the control facility 14 can, on the basis of the correction commands C, relocate the marking 9 to a section which is indicated as a broken line 10 in FIG. 2. Even though this is not explicitly represented in FIG. 2, the image 11 is where appropriate relocated at the same time.

In a step S8, the control facility 14 checks whether it receives a close command T from the operator 13. If and as long as this is not the case, the control facility 14 returns to step S6. In contrast, if this is the case, the control facility 14 skips to step S9. In step S9, and thus as a result of the specification of the close command T, the control facility 14 takes over as final the section onto which the marking 9 is last projected (i.e. during the specification of the close command T). The control facility 14 then moves the patient couch 6 in a step S10 such that the finally specified section is moved in the longitudinal direction x into the isocenter 3 of the modality 1. FIG. 5 shows this state purely by way of example.

A step S11 is often additionally present. If necessary, step S11 can be performed before or after step S10. In step S11, the control facility 14 terminates the actuation of the display facility 8.

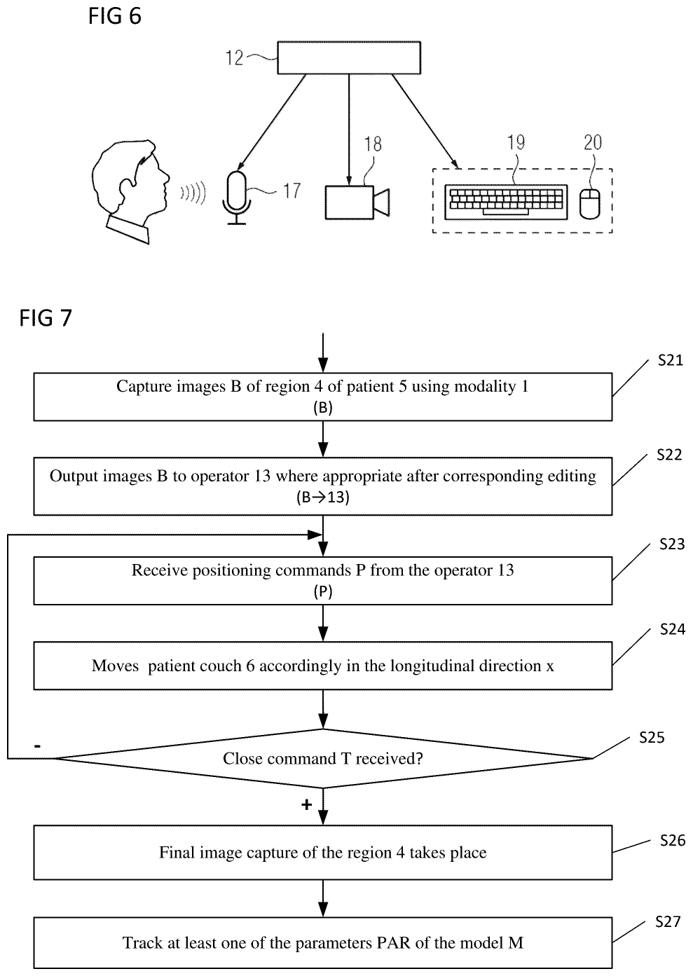

The specification of the correction commands C can take place as required. For example, the control facility 14 can execute the correction commands C as voice commands (a microphone 17 is indicated in FIG. 6 as a corresponding part of the interface 12), as gestures (a camera 18 is indicated in FIG. 6 as a corresponding part of the interface 12) or as manual inputs (a standard keyboard 19 and a standard mouse 20 are indicated in FIG. 6 as corresponding parts of the interface 12).

In a preferred aspect in accordance with FIG. 3 a step S12 follows step S10 or step S11. In step S12, the control facility 14 tracks at least one of the parameters PAR of the model M. The tracking takes place by utilizing the location of the final section of the patient couch 6, i.e. the point at which the marking 9 is positioned during specification of the close command T. Alternatively to the aforementioned point, in step S12 the difference between the section of the patient couch provisionally determined in step S4 and the final section of the patient couch 6 can be utilized.

Regardless of whether or not step S11 and/or step S12 are present, the control facility 14, in accordance with FIG. 7, generally executes steps S21 and S22 after step S10. In step S21, the control facility 14 captures a number of images B of the region 4 of the patient 5 (known as localizer acquisitions) by means of the modality 1. In step S22, the control facility 14 outputs the images B to the operator 13—where appropriate after corresponding editing.

Steps S23 to S26 preferably follow step S22. In step S23, the control facility 14 receives positioning commands P from the operator 13. The specification of the positioning commands P can be analogous to the specification of the correction commands C. In step S24, the control facility 14 moves the patient couch 6 accordingly in the longitudinal direction x. In step S25, the control facility 14 checks whether it receives a close command T from the operator 13. If and as long as this is not the case, the control facility 14 returns to step S23. In contrast, if this is the case, the control facility 14 skips to step S26. In step S26, the final image capture of the region 4, i.e., the diagnostic image capture of the region 4, takes place.

Where appropriate, a step S27 can additionally be present. If step S27 is present, the control facility 14 tracks at least one of the parameters PAR of the model M by utilizing the extent to which it moves the patient couch 6 in the longitudinal direction x on the basis of the positioning commands P, or the point to which it moves the patient couch 6 in the longitudinal direction x. Step S27 can be implemented analogously to step S12.

The present disclosure has many advantages. In particular, it provides a superior way of positioning the patient couch 6 such that the selected region 4 of the patient 5 can be moved (correctly) into the isocenter 3. Furthermore, the model M—possibly with the aid of a database—can be gradually and continuously improved. The present disclosure can also be employed with all types of medical imaging systems 1 in which a movement of the selected region 4 into the isocenter 3 takes place by means of a patient couch 6. However, its use is particularly advantageous in the case of corresponding medical imaging systems 1, which work with ionizing radiation. This is because the radiation dose to which the patient 5 is exposed can often be reduced by the inventive procedure because incorrect measurements can be prevented.

1. An operating method for a medical imaging system having a patient couch that is movable in a longitudinal direction such that different regions of a patient lying on the patient couch are movable in the longitudinal direction into an isocenter of the medical imaging system, the operating method comprising:
a control facility of the medical imaging system receiving from an operator data which characterizes properties of the patient him/herself and the approximate arrangement of the patient on the patient couch;
the control facility receiving a selection command from the operator for a selection of a region of the patient which is to be examined using the medical imaging system;
the control facility utilizing the data about the patient, viewed in the longitudinal direction of the patient couch, to provisionally determine a section of the patient couch at which the selected region of the patient is assumed;
the control facility actuating a display facility, so that a marking is projected onto the provisionally determined section of the patient couch;
based on correction commands which it receives from the operator, the control facility relocating the marking in the longitudinal direction, until it receives a close command from the operator; and
based on the specification of the close command, the control facility accepting as final the section onto which the marking is projected during specification of the close command, and moving the patient couch such that the final section is moved in the longitudinal direction into the isocenter of the medical imaging system.

2. The operating method as claimed in claim 1, wherein the marking comprises a line running transversely to the longitudinal direction of the patient couch.

3. The operating method as claimed in claim 1, wherein the marking comprises an image of the selected region of the patient.

4. The operating method as claimed in claim 3, further comprising:
the control facility ascertaining the size of the image as a function of the data characterizing the properties of the patient him/herself.

5. The operating method as claimed in claim 1, further comprising:
the control facility receiving the correction commands as voice commands, as gestures, or as manual inputs.

6. The operating method as claimed in claim 1, further comprising:
due to the specification of the close command, the control facility terminating the actuation of the display facility.

7. The operating method as claimed in claim 1, further comprising:
the control facility utilizing a model of the patient in addition to the data about the patient in connection with the provisional determination of the section of the patient couch, wherein the model is parameterized with parameters; and
the control facility tracking at least one of the parameters of the model by utilizing the location of the final section of the patient couch or the difference between the provisionally determined section of the patient couch and the final section of the patient couch.

8. The operating method as claimed in claim 1, further comprising:
the control facility, after moving the patient couch such that the final section is moved in the longitudinal direction into the isocenter of the medical imaging system, capturing a number of images of the region of the patient using the medical imaging system and outputting them to the operator; and
the control facility then receiving positioning commands from the operator, on the basis of which it moves the patient couch in the longitudinal direction.

9. The operating method as claimed in claim 8, further comprising:
the control facility, in connection with the provisional determination of the section of the patient couch, utilizing a model of the patient in addition to the data about the patient, wherein the model is parameterized with parameters; and
the control facility tracking at least one of the parameters of the model by utilizing the extent to which it moves the patient couch in the longitudinal direction on the basis of the positioning commands, or the point to which it moves the patient couch in the longitudinal direction.

10. A non-transitory computer-readable storage medium having a control program for a software-programmable control facility for a medical imaging system, wherein the control program comprises machine code, wherein the processing of the machine code by the control facility causes the control facility to execute an operating method as claimed in claim 1.

11. A medical imaging system, comprising:
a patient couch that is movable in a longitudinal direction such that different regions of a patient lying on the patient couch are movable in the longitudinal direction into an isocenter of the medical imaging system;
a display facility, by which a marking is projectable onto a section of the patient couch;
an interface to an operator; and
a control facility with a data connection to the patient couch, the display facility, and the interface, wherein the control facility is designed to control the medical imaging system in accordance with an operating method as claimed in claim 1.
